(19) United States
(12) Patent Application Publication
(10) Pub. No.: US 2006/0280644 A1
(43) Pub. Date: Dec. 14, 2006

(54) Method for terminal sterilization of transdermal delivery devices

(76) Inventors: Scott Sellers, San Mateo, CA (US); Yuh-Fun Maa, Millbrae, CA (US)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 11/446,530

(22) Filed: Jun. 1, 2006

Related U.S. Application Data
(60) Provisional application No. 60/687,519, filed on Jun. 2, 2005.

(51) Int. Cl. A61L 2/00

(52) U.S. Cl. 422/22

(57) ABSTRACT

A method and system for providing a terminally sterilized transdermal influenza vaccine delivery device. A microprojection member having a plurality of stratum corneum-piercing microprojections is coated with an influenza vaccine-formulation and exposed to sufficient radiation to sterilize the microprojection member while retaining sufficient potency of the influenza vaccine. Preferably, the microprojection member is sealed in packaging, such as a foil pouch. Also preferably, a retainer ring and adhesive are included within the packaging. The sterilizing radiation can be gamma radiation or e-beam, preferably delivered in a dose in the range of approximately 7-21 kGy. Also preferably, the irradiation is performed from −78.5-25° C. In preferred embodiments, the radiation is delivered at a rate greater than 3.0 kGy/hr.

### CROSS-REFERENCE TO RELATED APPLICATIONS

This application claims the benefit of U.S. Provisional Application No. 60/687,519, filed Jun. 2, 2005.

FIELD OF THE PRESENT INVENTION

The present invention relates generally to transdermal agent delivery systems and methods. More particularly, the invention relates to methods for sterilizing a transdermal device adapted to deliver an influenza vaccine.

### BACKGROUND OF THE INVENTION

Influenza presents a challenging public health concern, generally requiring specific vaccines to be design for each strain of virus expected. The influenza virus exhibits unpredictable changes of the surface glycoproteins, hemagglutinin and neuraminidase, leading to varying antigenic activity. These changes eventually lead to new influenza strains.

Immunization towards influenza virus is limited by this marked antigenic variation of the virus and by the restriction of the infection to the respiratory mucous membranes. The influenza vaccines currently available and licensed are based either on whole inactive virus, or on viral surface glycoproteins.

Influenza virus comprises two surface antigens: neuraminidase and hemagglutinin, which undergo changes leading to the high antigenic variations in influenza. Hemagglutinin is a strong immunogen and is the most significant antigen in defining the serological specificity of the different virus strains. The hemagglutinin molecule (75-80 kD) comprises a plurality of antigenic determinants, several of which are in regions that undergo sequence changes in different strains (strain-specific determinants) and others in regions which are common to many HA molecules (common determinants). Accordingly, hemagglutinin provides a useful basis for the formation of effective influenza vaccines.

As is well known in the art, skin is not only a physical barrier that shields the body from external hazards, but is also an integral part of the immune system. The immune function of the skin arises from a collection of residential cellular and humeral constituents of the viable epidermis and dermis with both innate and acquired immune functions, collectively known as the skin immune system.

One of the most important components of the skin immune system are the Langerhan's cells (LC), which are specialized antigen presenting cells found in the viable epidermis. LC's form a semi-continuous network in the viable epidermis due to the extensive branching of their dendrites between the surrounding cells. The normal function of the LC's is to detect, capture and present antigens to evoke an immune response to invading pathogens. LC's perform his function by internalizing epicutaneous antigens, trafficking to regional skin-draining lymph nodes, and presenting processed antigens to T cells.

The effectiveness of the skin immune system is responsible for the success and safety of vaccination strategies that have been targeted to the skin. Vaccination with a live-attenuated smallpox vaccine by skin scarification has successfully led to global eradication of the deadly small pox disease. Intradermal injection using ⅕ to 1/10 of the standard IM doses of various vaccines has been effective in inducing immune responses with a number of vaccines.

Transdermal delivery is thus a viable alternative for administering active agents, particularly, hemagglutinin antigen, that would otherwise need to be delivered via hypodermic injection or intravenous infusion. The word “transdermal”, as used herein, is a generic term that refers to delivery of an active agent (e.g., a therapeutic agent, such as a protein or an immunologically active agent, such as a vaccine) through the skin to the local tissue or systemic circulatory system without substantial cutting or penetration of the skin, such as cutting with a surgical knife or piercing the skin with a hypodermic needle. Transdermal agent delivery thus includes intracutaneous, intradermal and intraepidermal delivery via passive diffusion as well as delivery based upon external energy sources, such as electricity (e.g., iontophoresis) and ultrasound (e.g., phonophoresis).

Passive transdermal agent delivery systems, which are more common, typically include a drug reservoir that contains a high concentration of an active agent. The reservoir is adapted to contact the skin, which enables the agent to diffuse through the skin and into the body tissues or bloodstream of a patient.

As is well known in the art, the transdermal drug flux is dependent upon the condition of the skin, the size and physical/chemical properties of the drug molecule, and the concentration gradient across the skin. Because of the low permeability of the skin to many drugs, transdermal delivery has had limited applications. This low permeability is attributed primarily to the stratum comeum, the outermost skin layer which consists of flat, dead cells filled with keratin fibers (i.e., keratinocytes) surrounded by lipid bilayers. This highly-ordered structure of the lipid bilayers confers a relatively impermeable character to the stratum comeum.

One common method of increasing the passive transdermal diffusional agent flux involves mechanically penetrating the outermost skin layer(s) to create micropathways in the skin. There have been many techniques and devices developed to mechanically penetrate or disrupt the outermost skin layers to create pathways into the skin. Illustrative is the drug delivery device disclosed in U.S. Pat. No. 3,964,482.

Other systems and apparatus that employ tiny skin piercing elements to enhance transdermal agent delivery are disclosed in U.S. Pat. Nos. 5,879,326, 3,814,097, 5,250,023, 3,964,482, Reissue No. 25,637, and PCT Publication Nos. WO 96/37155, WO 96/37256, WO 96/17648, WO 97/03718, WO 98/11937, WO 98/00193, WO 97/48440, WO 97/48441, WO 97/48442, WO 98/00193, WO 99/64580, WO 98/28037, WO 98/29298, and WO 98/29365; all incorporated herein by reference in their entirety.

The disclosed systems and apparatus employ piercing elements of various shapes and sizes to pierce the outermost layer (i.e., the stratum comeum) of the skin. The piercing elements disclosed in these references generally extend perpendicularly from a thin, flat member, such as a pad or sheet. The piercing elements in some of these devices are extremely small, some having a microprojection length of only about 25-400 microns and a microprojection thickness of only about 5-50 microns. These tiny piercing/cutting elements make correspondingly small microslits/microcuts in the stratum corneum for enhancing transdermal agent delivery therethrough.

The disclosed systems further typically include a reservoir for holding the agent and also a delivery system to transfer the agent from the reservoir through the stratum corneum, such as by hollow tines of the device itself. One example of such a device is disclosed in WO 93/17754, which has a liquid agent reservoir. The reservoir must, however, be pressurized to force the liquid agent through the tiny tubular elements and into the skin. Disadvantages of such devices include the added complication and expense for adding a pressurizable liquid reservoir and complications due to the presence of a pressure-driven delivery system.

As disclosed in U.S. patent application Ser. No. 10/045,842, which is fully incorporated by reference herein, it is also possible to have the active agent that is to be delivered coated on the microprojections instead of contained in a physical reservoir. This eliminates the necessity of a separate physical reservoir and developing an agent formulation or composition specifically for the reservoir.

As stated, hemagglutinin antigen is at present delivered solely via intravenous routes. It would thus be desirable to provide an agent delivery system that facilitates transdermal administration of influenza vaccine.

Parenteral pharmaceutical products such as hemagglutinin antigen must meet stringent standards of sterility. One conventional method for assuring a sterile product is aseptic manufacturing. However, the demands of maintaining a sterile environment throughout the manufacturing process are time-consuming, laborious, and extremely expensive.

A potentially attractive alternative to aseptic manufacturing is to sterilize the product at the end of the manufacturing process. Terminal sterilization is used routinely for stable small molecules. Unfortunately, this method presents major challenges for more labile biopharmaceutical products. In particular, complex biological molecular structures such as hemagglutinin antigen must be protected from degradation to retain therapeutic activity.

In U.S. Pat. Nos. 6,346,216 and 6,171,549, Kent discloses the use of low irradiation rates for the sterilization of various biological molecules. However, these teachings fail to address specific conditions tailored for vaccines or for transdermal delivery devices. Kent also fails to provide any discussion regarding the effect of packaging on the product's stability and focuses on irradiation at room temperature.

It is therefore an object of the present invention to provide a method for conveniently sterilizing a transdermal device adapted to deliver an influenza vaccine.

It is yet another object of the present invention to provide a method for sterilizing a transderrnal delivery system that is more cost efficient than aseptic manufacturing.

Another object of the present invention is to provide a method for terminal sterilization of an influenza vaccine adapted for transdermal delivery.

It is another object of the present invention to provide packaging conditions for a transdermal delivery device that are adapted to optimize stability of an influenza vaccine during sterilization.

Yet another object of the invention is to provide a method for terminally sterilizing a transdermal device for delivering an influenza vaccine wherein the vaccine retains a substantial degree of activity.

### SUMMARY OF THE INVENTION

In accordance with the above objects and those that will be mentioned and will become apparent below, the method and system for terminally sterilizing a transdermal influenza vaccine delivery device comprises the steps of providing a microprojection member and exposing the microprojection member to radiation selected from the group consisting of gamma radiation and e-beam, wherein the radiation is sufficient to reach a desired sterility assurance level. The microprojection member includes a plurality of stratum comeum-piercing microprojections with a biocompatible coating having at least one influenza vaccine disposed thereon. Preferably, the microprojection member is sealed within packaging adapted to protect the vaccine during irradiation. In one embodiment, the packing comprises a foil pouch.

In one aspect of the invention, the microprojection member is mounted on a retainer ring prior to sealing the microprojection member inside the packaging. In a preferred embodiment, both a retainer ring and adhesive are included within the sealed packaging.

The invention also comprises reducing the degradation of the influenza vaccine during sterilization by adjusting the temperature at which the irradiation occurs. In one embodiment, the microprojection member is irradiated at a temperature in the range of approximately −78.5 to 25° C. The microprojection members can be irradiated at a temperature of −78.5° C. under dry ice conditions. In another embodiment, the microprojection member is irradiated at a temperature in the range of approximately 0-25° C. In another embodiment, the microprojection member is irradiated at an ambient temperature in the range of approximately 20-25° C.

According to the invention, the microprojection member receives a dose of radiation that is approximately 7 kGy. In another embodiment, the dose is approximately 14 kGy. In yet another embodiment, the dose is approximately 21 kGy.

In another embodiment, the invention includes exposing the microprojection member to radiation at a rate of greater than approximately 3.0 kGy/hr.

In further embodiments of the invention, the microprojection member is exposed to sufficient radiation to achieve a sterility assurance level of 10⁻³.

In additional embodiments, the invention is a transdermal influenza vaccine delivery system, comprising a microprojection member including a plurality of microprojections that are adapted to pierce the stratum corneum of a patient having a biocompatible coating disposed on the microprojection member, the coating being formed from a coating formulation having at least one influenza vaccine and packaging adapted to protect the vaccine sealed around the microprojection member, wherein the sealed package has been exposed to radiation to sterilize the microprojection member. In one embodiment, the packaging comprises a foil pouch. Preferably, an adhesive is sealed inside the packaging with the microprojection member. Also preferably, the microprojection member is mounted on a retainer ring.

In additional embodiments, the invention is a transdermal system adapted to deliver an influenza vaccine, comprising a microprojection member including a plurality of microprojections that are adapted to pierce the stratum corneum of a patient, a hydrogel formulation having at least one influenza vaccine in communication with the microprojection member, and packaging adapted to protect the vaccine sealed around the microprojection member, wherein the sealed package has been exposed to radiation to sterilize the microprojection member.

In other embodiments, the invention is a transdermal system adapted to deliver an influenza vaccine, comprising a microprojection member including a plurality of microprojections that are adapted to pierce the stratum corneum of a patient, a solid film having at least one influenza vaccine disposed proximate to the microprojection member, and packaging adapted to protect the vaccine sealed around the microprojection member, wherein the sealed package has been exposed to radiation to sterilize the microprojection member. Preferably, the solid film made by casting a liquid formulation comprising at least one influenza vaccine, a polymeric material, a plasticizing agent, a surfactant and a volatile solvent.

In one embodiment of the invention, the microprojection member has a microprojection density of at least approximately 10 microprojections/cm², more preferably, in the range of at least approximately 200 -2000 microprojections/cm².

In one embodiment, the microprojection member is constructed out of stainless steel, titanium, nickel titanium alloys, or similar biocompatible materials.

In another embodiment, the microprojection member is constructed out of a non-conductive material, such as polymeric materials.

Alternatively, the microprojection member can be coated with a non-conductive material, such as Parylene®, or a hydrophobic material, such as Teflon®, silicon or other low energy material.

The coating formulations applied to the microprojection member to form solid biocompatible coatings can comprise aqueous and non-aqueous formulations. In at least one embodiment of the invention, the formulation(s) includes at least one influenza vaccine, which can be dissolved within a biocompatible carrier or suspended within the carrier.

Preferably, the influenza vaccine is a trivalent influenza vaccine. For example, the HA content of each strain in the trivalent vaccine is typically set at 15 μg for a single human dose, i.e., 45 μg total HA.

In one embodiment, the system is adapted to deliver 45μg of hemagglutinin to the APC-abundant epidermal layer, wherein at least 70% of the influenza vaccine is delivered to the noted epidermal layer.

### BRIEF DESCRIPTION OF THE DRAWINGS

Further features and advantages will become apparent from the following and more particular description of the preferred embodiments of the invention, as illustrated in the accompanying drawings, and in which like referenced characters generally refer to the same parts or elements throughout the views, and in which:

FIG. 1 is a perspective view of a portion of one example of a microprojection member;

FIG. 2 is a perspective view of the microprojection member shown in FIG. 1 having a coating deposited on the microprojections, according to the invention;

FIG. 3 is a side sectional view of a retainer having a microprojection member disposed therein, according to the invention;

FIG. 4 is a perspective view of the retainer shown in FIG. 3;

FIGS. 5-7 are representations of micrographs showing coating morphology following irradiation, according to the invention;

FIG. 8 is a graph illustrating hemagglutinin potency after varying gamma irradiation levels and temperatures, according to the invention;

FIG. 9 is a graph illustrating hemagglutinin potency after varying e-beam irradiation levels and temperatures, according to the invention

FIG. 10 is a graph illustrating total protein content of irradiated hemagglutinin at varying temperatures, according to the invention;

FIGS. 11-13 are representations of micrographs showing coating morphology following gamma irradiation, according to the invention;

FIG. 14 is a graph illustrating protein content after varying irradiation doses under selected environmental conditions, according to the invention;

FIG. 15 is a graph illustrating hemagglutinin potency following irradiation under selected environmental conditions, according to the invention;

FIGS. 16 and 17 are representations of micrographs showing coating morphology following ethylene oxide sterilization, according to the invention; and

FIG. 18 is a graph illustrating protein content after irradiation with various system components, according to the invention.

### DETAILED DESCRIPTION OF THE INVENTION

Before describing the present invention in detail, it is to be understood that this invention is not limited to particularly exemplified materials, methods or structures as such may, of course, vary. Thus, although a number of materials and methods similar or equivalent to those described herein can be used in the practice of the present invention, the preferred materials and methods are described herein.

It is also to be understood that the terminology used herein is for the purpose of describing particular embodiments of the invention only and is not intended to be limiting.

Unless defined otherwise, all technical and scientific terms used herein have the same meaning as commonly understood by one having ordinary skill in the art to which the invention pertains.

Further, all publications, patents and patent applications cited herein, whether supra or infra, are hereby incorporated by reference in their entirety.

Finally, as used in this specification and the appended claims, the singular forms “a, “an” and “the” include plural referents unless the content clearly dictates otherwise. Thus, for example, reference to “an antigen” includes two or more such antigens; reference to “a microprojection” includes two or more such microprojections and the like.

### Definitions

The term “transdermal”, as used herein, means the delivery of an agent into and/or through the skin for local or systemic therapy. The term “transdermal” thus means and includes intracutaneous, intradermal and intraepidermal delivery of an agent, such as a vaccine, into and/or through the skin via passive diffusion as well as energy-based diffusional delivery, such as iontophoresis and phonophoresis.

The term “transdermal flux”, as used herein, means the rate of transdermal delivery.

The term “influenza vaccine”, as used herein, refers to an active agent that fosters an immune response to one or more antigens associated with an influenza virus. Preferably, the influenza vaccine comprises a split-varion vaccine. More preferably, the influenza vaccine comprises one or more monovalent hemagglutinin antigens. Even more preferably, the vaccine is a trivalent influenza vaccine.

The term “co-delivering”, as used herein, means that a supplemental agent(s) is administered transdermally either before the influenza vaccine is delivered, before and during transdermal flux of the influenza vaccine, during transdermal flux of the influenza vaccine, during and after transdermal flux of the influenza vaccine, and/or after transdermal flux of the influenza vaccine. Additionally, two or more influenza vaccines may be formulated in the coatings and/or hydrogel formulation, resulting in co-delivery of the influenza vaccines.

It is to be understood that more than one influenza vaccine can be incorporated into the agent source, formulations, and/or coatings and/or solid film formulations of this invention, and that the use of the term “influenza vaccine” in no way excludes the use of two or more such antigens.

The term “microprojections” or “microprotrusions”, as used herein, refers to piercing elements which are adapted to pierce or cut through the stratum comeum into the underlying epidermis layer, or epidermis and dermis layers, of the skin of a living animal, particularly, a mammal and, more particularly, a human.

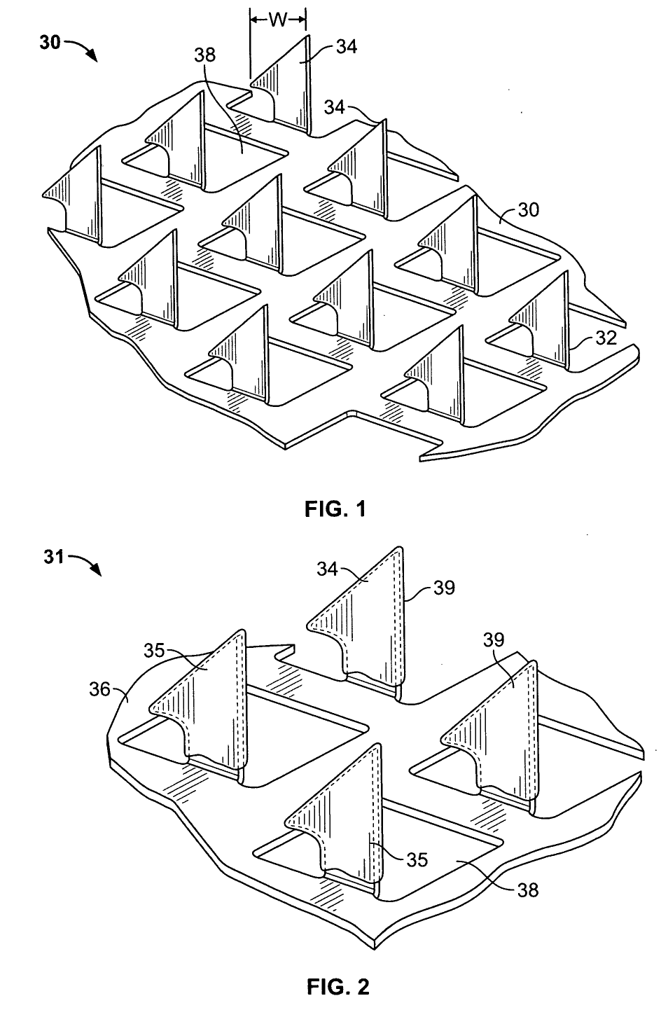

In one embodiment of the invention, the piercing elements have a projection length less than 1000 microns. In a further embodiment, the piercing elements have a projection length of less than 500 microns, more preferably, less than 250 microns. The microprojections further have a width (designated “W” in FIG. 1) in the range of approximately 25-500 microns and a thickness in the range of approximately 10-100 microns. The microprojections may be formed in different shapes, such as needles, blades, pins, punches, and combinations thereof.

The term “microprojection member”, as used herein, generally connotes a microprojection array comprising a plurality of microprojections arranged in an array for piercing the stratum comeum. The microprojection member can be formed by etching or punching a plurality of microprojections from a thin sheet and folding or bending the microprojections out of the plane of the sheet to form a configuration, such as that shown in FIG. 1. The microprojection member can also be formed in other known manners, such as by forming one or more strips having microprojections along an edge of each of the strip(s) as disclosed in U.S. Pat. No. 6,050,988, which is hereby incorporated by reference in its entirety.

The term “coating formulation”, as used herein, is meant to mean and include a freely flowing composition or mixture that is employed to coat the microprojections and/or arrays thereof. The influenza vaccine, if disposed therein, can be in solution or suspension in the formulation.

The term “biocompatible coating” and “solid coating”, as used herein, is meant to mean and include a “coating formulation” in a substantially solid form.

The term “biologically effective amount” or “biologically effective rate”, as used herein, refers to the amount or rate of the immunologically active agent needed to stimulate or initiate the desired immunologic, often beneficial result. The amount of the immunologically active agent employed in the coatings of the invention will be that amount necessary to deliver an amount of the immunologically active agent needed to achieve the desired immunological result. In practice, this will vary widely depending upon the particular immunologically active agent being delivered, the site of delivery, and the dissolution and release kinetics for delivery of the immunologically active agent into skin tissues.

The term “adhesive”, as used herein, is meant to mean and include an adhesive for helping maintain the microprojection member in place on a patient. Generally, the adhesive is in the form of a patch.

As indicated above, the present invention generally comprises a method for sterilizing a transdermal delivery system at the end of the manufacturing process. The invention also comprises the sterilized delivery systems. The transdermal delivery system includes a microprojection member (or system) having a plurality of microprojections (or array thereof) that are adapted to pierce through the stratum comeum into the underlying epidermis layer, or epidermis and dermis layers. The microprojection member (or system) also includes at least one source or delivery medium of influenza vaccine (i.e., biocompatible coating, hydrogel formulation and solid film formulation). The transdermal delivery system is terminally sterilized by exposure to sufficient radiation to achieve a desired sterility assurance level.

Gamma radiation can be delivered by conventional methods, such as by using Cobalt-60 as a radiation source. As one having skill in the art will recognize, a commercial Cobalt-60 sterilizer yields a rate of irradiation in the range of approximately 0.3 Gy/hr and 9.6 kGy/hr. Americium-241 can also be used, and generally irradiate at a rate of approximately 0.3 mGy/hr. Other isotopes can also be used to deliver gamma radiation at a desired rate. E-beam radiation is conventionally generated at substantially higher rates than gamma radiation, such as approximately 100 kGy/hr. In preferred embodiments, the dose rate is 3.0 kGy/hr or greater to minimize the processing time required to achieve a dose sufficient to reach the desired level of sterility.

The radiation dose required for terminal sterilization can be determined by conventional methods based upon the desired sterility assurance level (SAL) in relation to the bioburden of device being sterilized. For example, delivery systems of conventional parenteral active pharmaceutical agents typically require a SAL of 10⁻⁶. In other embodiments of the invention, a relatively low bioburden can be assigned to influenza vaccines because antigenic agents are typically evaluated by bioassays, as opposed to more stringent chromatographic methods. In the noted embodiments, a SAL of 10⁻³ can be used to tailor the radiation dose. As discussed below, the reduced sterility requirements allow lower doses of radiation for the terminal sterilization process which helps maintain the antigenicity of the influenza vaccine.

Thus, terminal sterilization of the microprojection member loaded with influenza vaccine is achieved by irradiating the system with e-beam or gamma irradiation. Suitable doses are in the range of approximately 10 to 25 kGy. Preferably, the dose is at least approximately 7 kGy. More preferably, the dose is approximately 14 kGy. A dose of approximately 21 kGy can also be used according to the invention.

Preferably, the microprojection member is sealed in packaging adapted to protect the vaccine during sterilization. In one embodiment, the packaging is a foil pouch.

In further embodiments of the invention, the microprojection member is mounted on a retainer ring for use with an applicator prior to being sealed into the packaging.

In other embodiments of the invention, an adhesive is included inside the packaging.

In further embodiments of the invention, irradiation of the microprojection member is conducted at defined temperatures to stabilize the influenza vaccine. In one embodiment, the microprojection member is irradiated at a temperature in the range of approximately −78.5 to 25° C. The microprojection members can be irradiated at a temperature of −78.5° C. under dry ice conditions. In another embodiment, the microprojection member is irradiated at a temperature in the range of approximately 0-25° C. In another embodiment, the microprojection member is irradiated at an ambient temperature in the range of approximately 20-25° C.

Preferably, the influenza vaccine comprises a split-varion vaccine. More preferably, the influenza vaccine comprises one or more monovalent hemagglutinin antigens. Even more preferably, the vaccine is a trivalent influenza vaccine.

Additional information regarding the terminal sterilization of other biologically active agents can be found in co-pending U.S. application Ser. Nos. 60/687,636, filed Jun. 2, 2005, and 60/687,635, filed Jun. 2, 2005, which are hereby incorporated by reference in their entirety.

Referring now to FIGS. 1 and 2, there is shown one embodiment of a microprojection member 30 for use with the present invention. As illustrated in FIG. 1, the microprojection member 30 includes a microprojection array 32 having a plurality of microprojections 34. The microprojections 34 preferably extend at substantially a 90° angle from the sheet, which in the noted embodiment includes openings 38. In this embodiment, the microprojections 34 are formed by etching or punching a plurality of microprojections 34 from a thin metal sheet 36 and bending the microprojections 34 out of the plane of the sheet 36.

In one embodiment of the invention, the microprojection member 30 has a microprojection density of at least approximately 10 microprojections/cm², more preferably, in the range of at least approximately 200-2000 microprojections/cm². Preferably, the number of openings per unit area through which the vaccine passes is at least approximately 10 openings/cm² and less than about 2000 openings/cm².

As indicated, the microprojections 34 preferably have a projection length less than 1000 microns. In one embodiment, the microprojections 34 have a projection length of less than 500 microns, more preferably, less than 250 microns. The microprojections 34 also preferably have a width in the range of approximately 25-500 microns and thickness in the range of approximately 10-100 microns.

To enhance the biocompatibility of the microprojection member 30 (e.g., to minimize bleeding and irritation following application to the skin of a subject), in a further embodiment, the microprojections 34 preferably have a length less than 145 μm, more preferably, in the range of approximately 50-145 μm, even more preferably, in the range of approximately 70-140 μm. Further, the microprojection member 30 comprises an array preferably having a microprojection density greater than 100 microprojections/cm², more preferably, in the range of approximately 200-3000 microprojections/cm^{2.}

The microprojection member 30 can be manufactured from various metals, such as stainless steel, titanium, nickel titanium alloys, or similar biocompatible materials.

According to the invention, the microprojection member 30 can also be constructed out of a non-conductive material, such as a polymer.

Alternatively, the microprojection member can be coated with a non-conductive material, such as Parylene®, or a hydrophobic material, such as Teflon®, silicon or other low energy material. The noted hydrophobic materials and associated base (e.g., photoreist) layers are set forth in U.S. application No. 60/484,142, which is incorporated by reference herein.

Microprojection members that can be employed with the present invention include, but are not limited to, the members disclosed in U.S. Pat. Nos. 6,083,196, 6,050,988 and 6,091,975, which are incorporated by reference herein in their entirety.

Other microprojection members that can be employed with the present invention include members formed by etching silicon using silicon chip etching techniques or by molding plastic using etched micro-molds, such as the members disclosed U.S. Pat. No. 5,879,326, which is incorporated by reference herein in its entirety.

According to the invention, the influenza vaccine to be administered to a host can be contained in a biocompatible coating that is disposed on the microprojection member 30 or contained in a hydrogel formulation or contained in both the biocompatible coating and the hydrogel formulation. Preferably, the hydrogel formulations of the invention comprise water-based hydrogels. Hydrogels are preferred formulations because of their high water content and biocompatibility. Also preferably, the hydrogel is configured as a gel pack.

In a further embodiment, wherein the microprojection member includes an vaccine-containing solid film formulation, the influenza vaccine can be contained in the biocompatible coating, hydrogel formulation or solid film formulation, or in all three delivery mediums.

In one embodiment, the solid film made by casting a liquid formulation comprising at least one influenza vaccine, a polymeric material, such as hyroxyethyl starch, dextran, hydroxyethylcellulose (HEC), hydroxypropylmethylcellulose (HPMC), hydroxypropycellulose (HPC), methylcellulose (MC), hydroxyethylmethylcellulose (HEMC), ethylhydroxethylcellulose (EHEC), carboxymethylcellulose (CMC), poly(vinyl alcohol), poly(ethylene oxide), poly(2-hydroxyethymethacrylate), poly(n-vinyl pyrolidone) and pluronics, a plasticizing agent, such as glycerol, propylene glycol and polyethylene glycol, a surfactant, such as Tween 20 and Tween 80, and a volatile solvent, such as water, isopropanol, methanol and ethanol.

In one embodiment, the liquid formulation used to produce the solid film comprises: 0.1-20 wt. % influenza vaccine, 5-40 wt. % polymer, 5-40 wt. % plasticizer, 0-2 wt. % surfactant, and the balance of volatile solvent.

Following casting and subsequent evaporation of the solvent, a solid film is produced.

Preferably, the influenza vaccine is present in the liquid formulation used to produce the solid film at a concentration in the range of approximately 0.1-2 wt. %.

According to the invention, at least one influenza vaccine is contained in at least one of the aforementioned delivery mediums. The amount of the influenza vaccine that is employed in the delivery medium and, hence, microprojection system will be that amount necessary to deliver a therapeutically effective amount of the influenza vaccine to achieve the desired result. In practice, this will vary widely depending upon the particular influenza vaccine, the site of delivery, the severity of the condition, and the desired therapeutic effect.

In one embodiment, the microprojection member includes a biocompatible coating that contains at least one influenza vaccine, preferably, trivalent hemagglutinin. The microprojection member is terminally sterilized to a desired sterility assurance level. Upon piercing the stratum corneum layer of the skin, the vaccine-containing coating is dissolved by body fluid (intracellular fluids and extracellular fluids such as interstitial fluid) and released into the skin (i.e., bolus delivery) for systemic therapy.

Referring now to FIG. 2, there is shown a microprojection member 31 having microprojections 34 that include a biocompatible coating 35 of the influenza vaccine. According to the invention, the coating 35 can partially or completely cover each microprojection 34. For example, the coating 35 can be in a dry pattern coating on the microprojections 34. The coating 35 can also be applied before or after the microprojections 34 are formed.

According to the invention, the coating 35 can be applied to the microprojections 34 by a variety of known methods. Preferably, the coating is only applied to those portions the microprojection member 31 or microprojections 34 that pierce the skin (e.g., tips 39).

One such coating method comprises dip-coating. Dip-coating can be described as a means to coat the microprojections by partially or totally immersing the microprojections 34 into a coating solution. By use of a partial immersion technique, it is possible to limit the coating 35 to only the tips 39 of the microprojections 34.

A further coating method comprises roller coating, which employs a roller coating mechanism that similarly limits the coating 35 to the tips 39 of the microprojections 34. The roller coating method is disclosed in U.S. application Ser. No. 10/099,604 (Pub. No. 2002/0132054), which is incorporated by reference herein in its entirety. As discussed in detail in the noted application, the disclosed roller coating method provides a smooth coating that is not easily dislodged from the microprojections 34 during skin piercing.

According to the invention, the microprojections 34 can further include means adapted to receive and/or enhance the volume of the coating 35, such as apertures (not shown), grooves (not shown), surface irregularities (not shown) or similar modifications, wherein the means provides increased surface area upon which a greater amount of coating can be deposited.

A further coating method that can be employed within the scope of the present invention comprises spray coating. According to the invention, spray coating can encompass formation of an aerosol suspension of the coating composition. In one embodiment, an aerosol suspension having a droplet size of about 10 to 200 picoliters is sprayed onto the microprojections 34 and then dried.

Pattern coating can also be employed to coat the microprojections 34. The pattern coating can be applied using a dispensing system for positioning the deposited liquid onto the microprojection surface. The quantity of the deposited liquid is preferably in the range of 0.1 to 20 nanoliters/microprojection. Examples of suitable precision-metered liquid dispensers are disclosed in U.S. Pat. Nos. 5,916,524; 5,743,960; 5,741,554; and 5,738,728; which are fully incorporated by reference herein.

Microprojection coating formulations or solutions can also be applied using ink jet technology using known solenoid valve dispensers, optional fluid motive means and positioning means which is generally controlled by use of an electric field. Other liquid dispensing technology from the printing industry or similar liquid dispensing technology known in the art can be used for applying the pattern coating of this invention.

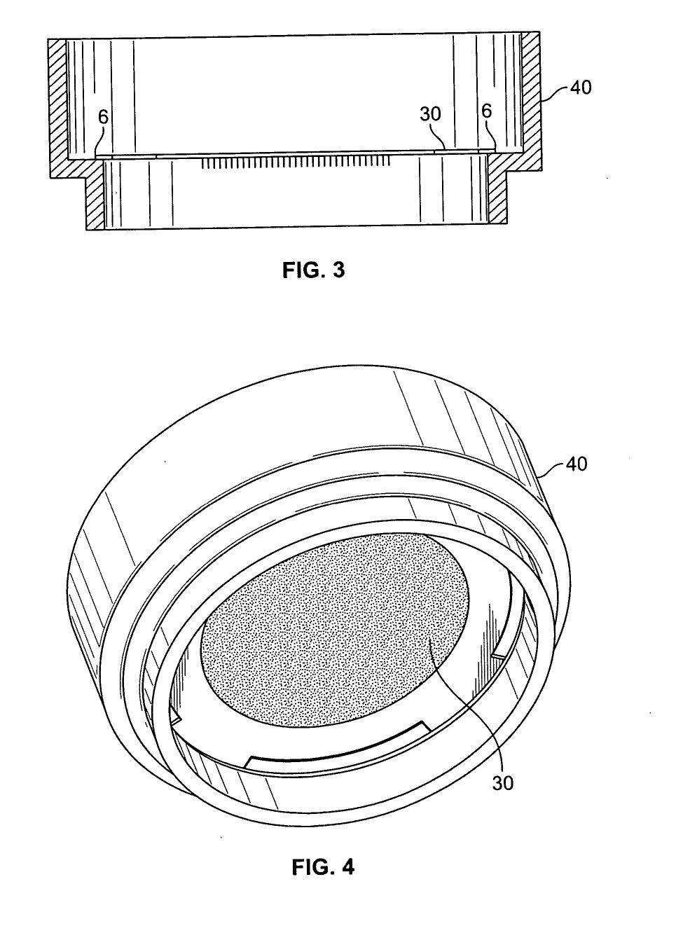

Referring now to FIGS. 3 and 4, for storage and application, the microprojection member 30 is preferably suspended in a retainer ring 40 by adhesive tabs 6, as described in detail in U.S. application Ser. No. 09/976,762 (Pub. No. 2002/0091357), which is incorporated by reference herein in its entirety.

After placement of the microprojection member in the retainer ring 40, the microprojection member is applied to the patient's skin. Preferably, the microprojection member is applied to the patient's skin using an impact applicator, as described in Co-Pending U.S. application Ser. No. 09/976,978, which is incorporated by reference herein in its entirety. As discussed above, retainer ring 40 is preferably pre-dried prior to packaging to reduce the amount of moisture in the atmosphere surrounding the microprojection member during irradiation.

As indicated, according to one embodiment of the invention, the coating formulations applied to the microprojection member 30 to form solid biocompatible coatings can comprise aqueous and non-aqueous formulations having at least one influenza vaccine. According to the invention, the influenza vaccine can be dissolved within a biocompatible carrier or suspended within the carrier.

As is well known in the art, the influenza virus particle consists of many protein components with hemagglutinin (HA) as the primary surface antigen responsible for the induction of protective anti-HA antibodies in humans. Immunologically, influenza A viruses are classified into subtypes on the basis of two surface antigens: HA and neuraminidase (NA). Immunity to these antigens, especially to the hemagglutinin, reduces the likelihood of infection of infection and lessens the severity of the disease if infection occurs.

The antigenic characteristics of circulating strains provide the basis for selecting the virus strains included in each year's vaccine. Every year, the influenza vaccine contains three virus strains (usually two type A and one B) that represent the influenza viruses that are likely to circulate worldwide in the coming winter. Influenza A and B can be distinguished by differences in their nucleoproteins and matrix proteins. Type A is the most common strain and is responsible for the major human pandemics. Accordingly, the influenza vaccine preferably comprises a trivalent influenza vaccine. For example, the HA content of each strain in the trivalent vaccine is typically set at 15 μg for a single human dose, i.e., 45 μg total HA.

In one embodiment, a full human dose of the influenza vaccine, i.e., 45 μg of hemagglutinin, can be transdermally delivered to the APC-abundant epidermal layer, the most immuno-competent component of the skin, via a coated microprojection array, wherein at least 70% of the influenza vaccine is delivered to the noted epidermal layer. More importantly, the antigen remains immunogenic in the skin to elicit strong antibody and sero-protective immune responses. Additional details regarding suitable influenza vaccine formulations can be found in co-pending U.S. application Ser. No. 11/084,631, filed Mar. 18, 2005, and Ser. No. 11/084,635, filed Mar. 18, 2005, which are hereby incorporated by reference in their entirety.

Suitable immune response augmenting adjuvants which, together with the vaccine antigen, can comprise the vaccine include, without limitation, aluminum phosphate gel; aluminum hydroxide; algal glucan: β-glucan; cholera toxin B subunit; CRL1005: ABA block polymer with mean values of x=8 and y=205; gamma inulin: linear (unbranched) β-D(2−>1) polyfructofuranoxyl-α-D-glucose; Gerbu adjuvant: N-acetylglucosamine-(β1-4)-N- acetylmuramyl-L-alanyl-D-glutamine (GMDP), dimethyl dioctadecylammonium chloride (DDA), zinc L-proline salt complex (Zn-Pro-8); Imiquimod (1-(2-methypropyl)-1H-imidazo[4,5-c]quinolin-4-amine; ImmTher™: N-acetylglucoaminyl-N-acetylmuramyl-L-Ala-D-isoGlu-L-Ala-glycerol dipalmitate; MTP-PE liposomes: C₅₉H₁₀₈N₆O₁₉PNa -3H₂O (MTP); Murametide: Nac-Mur-L-Ala-D-Gln-OCH₃; Pleuran: β-glucan; QS-21; S-28463: 4-amino-a, a-dimethyl-1H-imidazo[4,5-c]quinoline-1-ethanol; salvo peptide: VQGEESNDK•HCl (IL-1 163-171 peptide); and threonyl-MDP (Termurtide™): N-acetyl muramyl-L-threonyl-D-isoglutamine, and interleukine 18, IL-2 IL-12, IL-15, Adjuvants also include DNA oligonucleotides, such as, for example, CpG containing oligonucleotides. In addition, nucleic acid sequences encoding for immuno-regulatory lymphokines such as IL-18, IL-2 IL-12, IL-15, IL-4, IL10, gamma interferon, and NF kappa B regulatory signaling proteins can be used.

According to the invention, the amount and type of adjuvant can be adapted to optimize the stability of the influenza vaccine during sterilization.

Preferably, the coating formulations have a viscosity less than approximately 500 centipoise and greater than 3 centipose.

In one embodiment of the invention, the coating thickness is less than 25 microns, more preferably, less than 10 microns as measured from the microprojection surface.

The desired coating thickness is dependent upon several factors, including the required dosage and, hence, coating thickness necessary to deliver the dosage, the density of the microprojections per unit area of the sheet, the viscosity and concentration of the coating composition and the coating method chosen. The thickness of coating 35 applied to microprojections 34 can also be adapted to optimize stability of the influenza vaccine.

In all cases, after a coating has been applied, the coating formulation is dried onto the microprojections 34 by various means. In a preferred embodiment of the invention, the coated microprojection member 30 is dried in ambient room conditions. However, various temperatures and humidity levels can be used to dry the coating formulation onto the microprojections. Additionally, the coated member can be heated, stored under vacuum or over desiccant, lyophilized, freeze dried or similar techniques used to remove the residual water from the coating.

It will be appreciated by one having ordinary skill in the art that in order to facilitate drug transport across the skin barrier, the present invention can also be employed in conjunction with a wide variety of iontophoresis or electrotransport systems, as the invention is not limited in any way in this regard. Illustrative electrotransport drug delivery systems are disclosed in U.S. Pat. Nos. 5,147,296, 5,080,646, 5,169,382 and 5,169,383, the disclosures of which are incorporated by reference herein in their entirety.

The term “electrotransport” refers, in general, to the passage of a beneficial agent, e.g., a vaccine or a drug or drug precursor, through a body surface such as skin, mucous membranes, nails, and the like. The transport of the agent is induced or enhanced by the application of an electrical potential, which results in the application of electric current, which delivers or enhances delivery of the agent, or, for “reverse” electrotransport, samples or enhances sampling of the agent. The electrotransport of the agents into or out of the human body may by attained in various manners.

One widely used electrotransport process, iontophoresis, involves the electrically induced transport of charged ions. Electroosmosis, another type of electrotransport process involved in the transdernal transport of uncharged or neutrally charged molecules (e.g., transdermal sampling of glucose), involves the movement of a solvent with the agent through a membrane under the influence of an electric field. Electroporation, still another type of electrotransport, involves the passage of an agent through pores formed by applying an electrical pulse, a high voltage pulse, to a membrane.

In many instances, more than one of the noted processes may be occurring simultaneously to different extents. Accordingly, the term “electrotransport” is given herein its broadest possible interpretation, to include the electrically induced or enhanced transport of at least one charged or uncharged agent, or mixtures thereof, regardless of the specific mechanism(s) by which the agent is actually being transported. Additionally, other transport enhancing methods, such as sonophoresis or piezoelectric devices, can be used in conjunction with the invention.

### EXAMPLES

The following examples are given to enable those skilled in the art to more clearly understand and practice the present invention. They should not be considered as limiting the scope of the invention but merely as being illustrated as representative thereof.

### Example 1

Formulations of trivalent influenza vaccine were prepared and coated on microprojection arrays. The coated arrays were placed in scintillation glass vials for irradiation. The samples were subjected to gamma radiation and e-beam radiation doses of 7, 14 and 21 kGy under dry ice and at an ambient temperature. Hemagglutinin content in the coated arrays following irradiation was assessed using single radial immuno-diffusion assays (SRID) and bicinchoninic acid protein assays (BCA). SRID involves forming a zone of precipitation where the antigen and appropriate anti-sera interact. The formed zone is directly proportional to the amount of antigen present in the test preparation. The antigen to be tested was added to wells in an agarose gel containing the anti-sera. The antigen and anti-sera interact, diffuse and precipitate in zones around the wells. Coomassie Blue staining allowed visualization of the zone. Diameters of the tested antigen were then compared to reference standards to quantify quantify the amount of antigen. SRID is the only approved in vitro potency assay for the influenza vaccine. As those of skill in the art will recognize, hemagglutinin potency corelates well with immunogenicity.

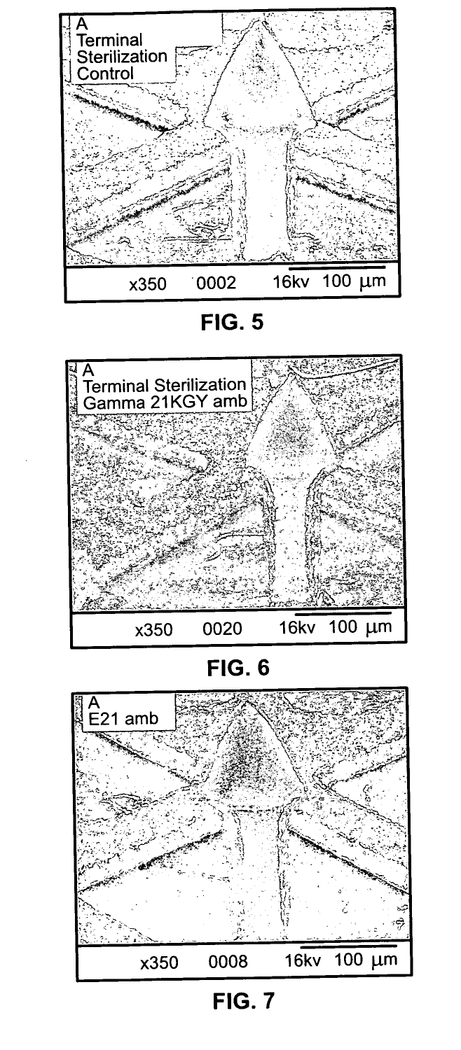

FIGS. 5-7 are representations of scanning electron micrographs illustrating the morphology of microprojection array tips coated with influenza vaccine. FIG. 5 shows the morphology of a control array that was not irradiated, while FIG. 6 and FIG. 7 show the tips of microprojection arrays that were irradiated with 21 kGy of gamma radiation and e-beam radiation, respectively. As can be seen, the shape and surface smoothness of the tips of the irradiated arrays was not substantially changed from the control array. This indicates the physical characteristics of the coating are not negatively affected by the sterilization radiation.

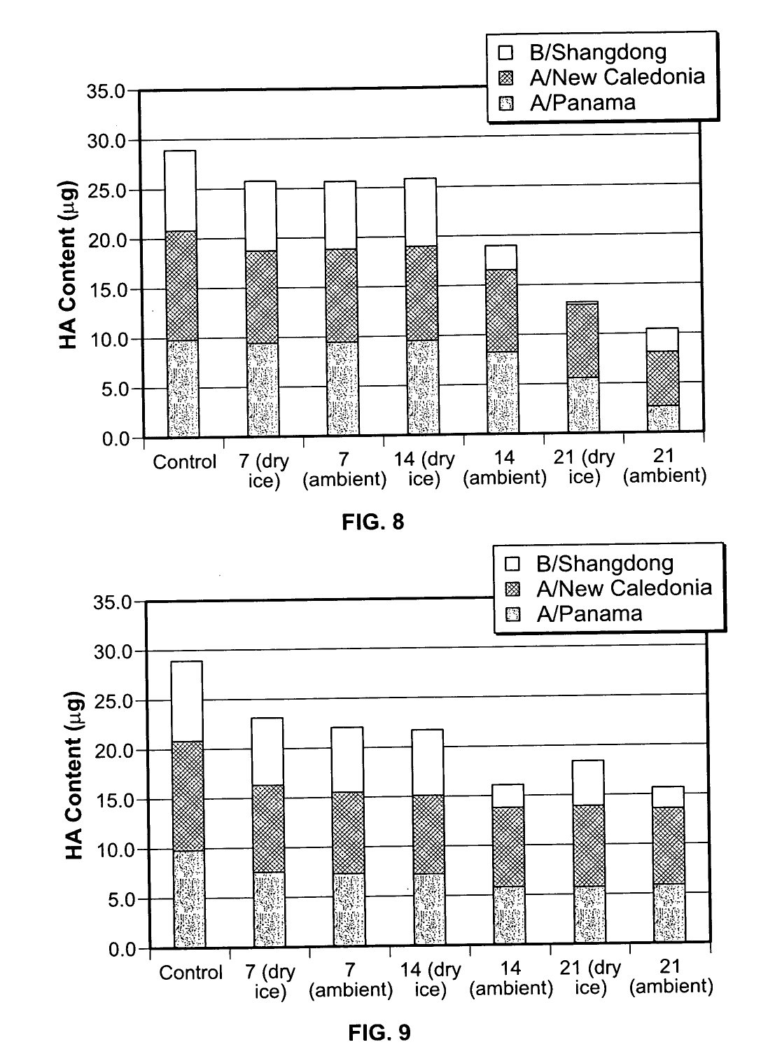

The SRID assay results for the irradiated microprojection arrays is shown in FIGS. 8 and 9, for gamma irradiation and e-beam, respectively. In general, both gamma and e-beam irradiation affected the influenza vaccine to approximately the same degree and reduced the potency of the hemagglutinin, particularly at the high radiation doses. Further, the B/Shangdong strain exhibited greater sensitivity to the sterilization procedure. These results also demonstrated that decreasing the irradiation dose helped preserve the hemagglutinin potency. For example, less than 20% potency loss was observed at 7 kGy. Additionally, this experiment demonstrated that lowered irradiation temperature reduces potency loss, with the best results obtained under dry ice.

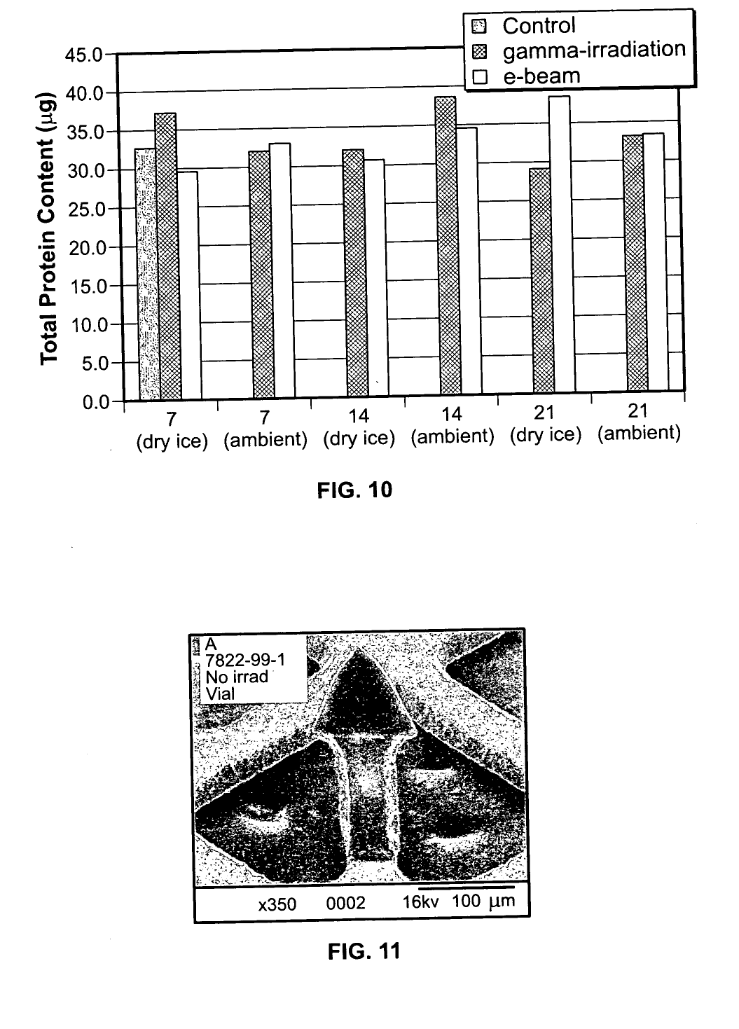

FIG. 10 shows the total protein content of the irradiated microprojection arrays. The BCA analysis also demonstrates the water solubility of the coating. As can be appreciated, attenuated solubility in conjunction with lowered protein content is indicative of significant chemical changes in the vaccine formulation. Notably, this study showed that the protein content in each of the samples was fully recovered. Accordingly, this is a good indication that the solubility of the vaccine coating was unchanged by the irradiation procedure.

### Example 2

Formulations of trivalent influenza vaccine were prepared and coated on microprojection arrays. The samples were subjected to gamma radiation and e-beam radiation doses of 7 and 14 kGy under dry ice and at ambient temperatures of 20-25° C. Certain microprojection arrays were assembled with polycarbonate retainer rings and an adhesive, then packaged in foil pouches. Hemagglutinin content in the coated arrays following irradiation was assessed using SRID and BCA.

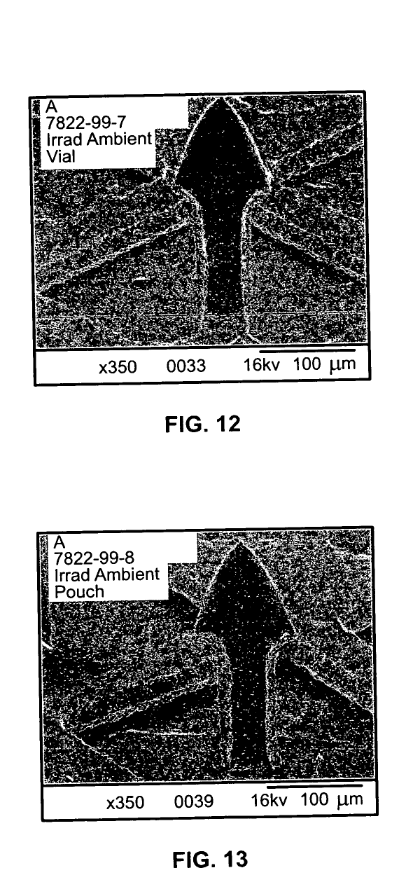

FIGS. 11-13 are representations of scanning electron micrographs illustrating the morphology of microprojection array tips coated with influenza vaccine. FIG. 11 shows the morphology of a control array that was not irradiated, while FIG. 6 and FIG. 7 show the tips of microprojection arrays that were irradiated with 14 kGy of gamma radiation in a glass vial and in a foil pouch, respectively. As can be seen, the shape and surface smoothness of the tips of the irradiated arrays was not substantially changed from the control array. The results corroborate those reported in Example 1, indicating the physical characteristics of the coating are not negatively affected by the sterilization radiation.

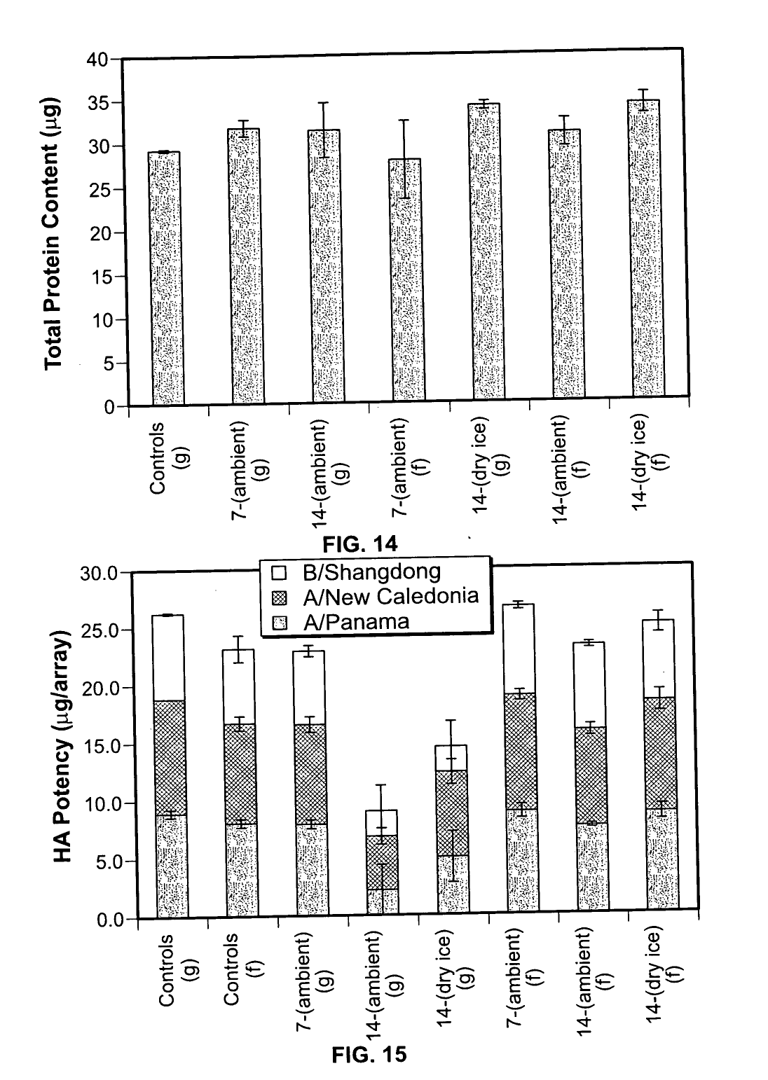

Further, FIG. 14 shows that protein recovery in this study was comparable to that of Example 1. Specifically, the BCA analysis indicated that the solubility of the vaccine coating was unchanged by the irradiation procedure.

The irradiated samples were also assayed by SRID, and the results are shown in FIG. 15. For the samples contained in glass vials, degradation at the 14 kGy dose was significant, with a 40% potency loss under dry ice and more than 50% at an ambient temperature. The effect of dose is marked, as the samples that received the 7 kGy dose suffered no significant potency loss. An important result shown is the retention of potency for the fully assembled and foil pouch packaged samples, even at the high dose of 14 kGy at ambient temperature. Accordingly, this example demonstrated that assembled and packaged arrays coated with influenza vaccine could be terminally sterilized effectively.

Example 3

As in Example 1, formulations of trivalent influenza vaccine were prepared and coated on microprojection arrays. The samples were subjected to gamma radiation doses of 7 and 14 kGyunder dry ice and at an ambient temperature. The microprojection arrayswere packaged with various components of the microprojection system to assess the impact of those components on the vaccine's stability during irradiation. One sample was subjected to ethylene oxide sterilization instead of radiation. Hemagglutinin content in the coated arrays following sterilization was assessed using SRID and BCA. The packaging and sterilization protocol for this example is given in Table 1.

TABLE 1

| | | | Irradiation | |
|---|---|---|---|---|
| Group | | | Dose | Irradiation |
| No. | Packaging | System Components | (kGy) | Temp. |
| 1 | Foil pouch | Ring, Adhesive | | |
| 2 | Foil pouch | Ring, Adhesive | 21 | 20-25° C. |
| 3 | Foil pouch | Ring, Adhesive | 21 | 20-25° C. |
| 4 | Foil pouch | Ring, Adhesive | 14 | 20-25° C. |
| 5 | Foil pouch | Adhesive | 14 | 20-25° C. |
| 6 | Foil pouch | Ring | 14 | 20-25° C. |
| 7 | Foil pouch | | 14 | 20-25° C. |
| 8 | Glass vial | | 14 | 20-25° C. |
| 9 | Glass vial | Adhesive | 14 | 20-25° C. |
| 10 | Glass vial | | EO | |

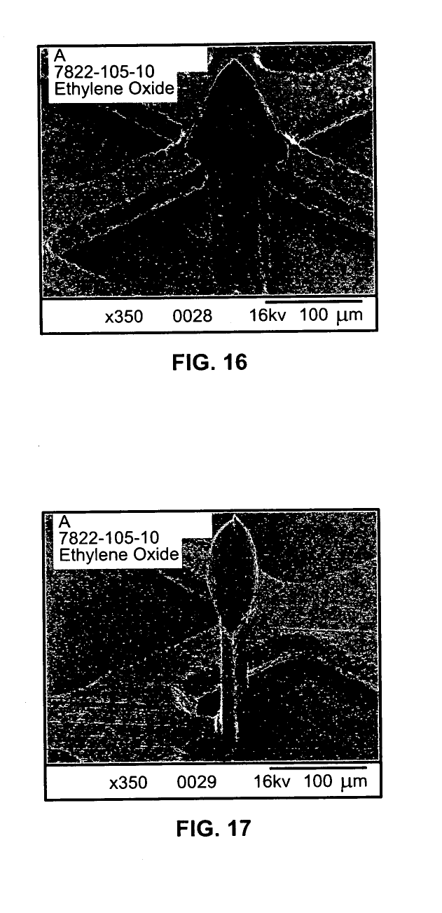

FIGS. 16 and 17 are representations of scanning electron micrographs illustrating different views of the coated microprojection array tips morphology of Group 10 after ethylene oxide sterilization. As shown, no significant detrimental effect was observed regarding the physical characteristics of the vaccine coating. In contrast, more hygroscopic pharmacological agents such as hPTH experience unacceptable morphological changes. Accordingly, active agent formulations having relatively low hygroscopicity, such as influenza vaccine, can be subjected to ethylene oxide sterilzation without significantly damaging the coating.

The BCA analysis of the samples in this study tracked the results obtained in the examples above, as the protein content in each system was fully recovered. As discused above, this indicates that the solubility of the vaccine coating was unchanged

The BCA analysis of the samples in this study tracked the results obtained in the examples above, as the protein content in each system was fully recovered. As discussed above, this indicates that the solubility of the vaccine coating was unchanged by the irradiation procedure. These findings similarly indicate that irradiation does not dramatically affect the chemical composition of the flu vaccine formulations.

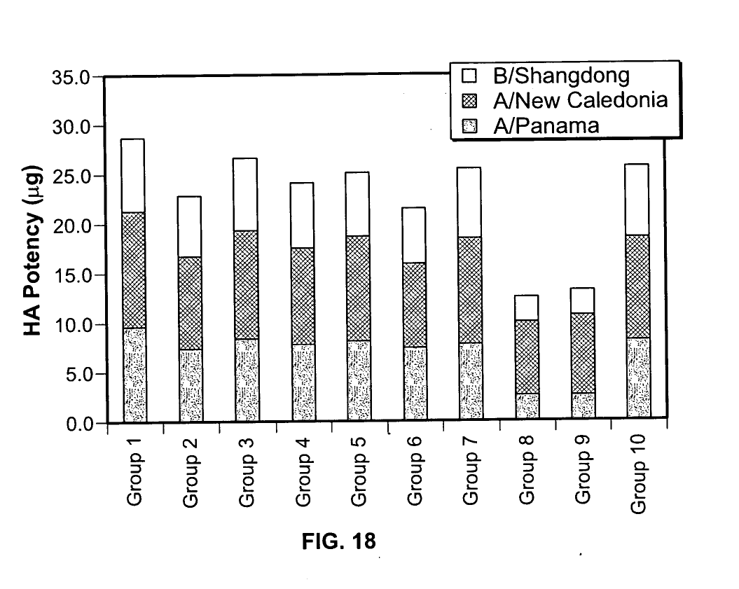

The irradiated samples were also assayed by SRID, and the results are shown in FIG. 18. This study demonstrated that fully packaged microprojection systems provided good potency retention, even at the high radiation dose of 21 kGy. Indeed, the potency retention for Group 3 under dry ice exhibited only minimal potency loss. This study also indicated improved results for foil pouch packaged arrays as opposed to glass vials. Specifically, Groups 8 and 9 experienced significant potency loss at 14 kGy doses, particularly for the B/Shangdong and A/Panama strains.

This example further indicates that the components of the microprojection system impact the stability of the flu vaccine during irradiation. As shown by the results for Groups 5-7, the foil pouch appears to provide the greatest protection, followed by the adhesive and then the retainer ring.

Also, the sample subjected to ethylene oxide sterilization retained essentially full potency. Thus, these results indicate that ethylene oxide can be used to effectively sterilize a transdermal flu vaccine delivery system without detrimentally affecting the physical characteristics or the hemagglutinin potency.

As shown by the above examples and discussion, microprojection members having a coating formulation including an influenza vaccine such as hemagglutinin antigen can be terminally sterilized by either gamma irradiation or e-beam treatment with little or no reduction in potency using the methods of the invention. Preferably, the packaging of the microprojection members is adapted to protect the vaccine during the terminal sterilization process. For example, a sealed foil pouch has a significant stabilizing effect. Also preferably, the microprojection member is mounted on a retainer ring and assembled with an adhesive prior to packaging.

Further, product degradation can also be reduced during the terminal sterilization process by adjusting the temperature or by reducing the sterilization dose.

Without departing from the spirit and scope of this invention, one of ordinary skill can make various changes and modifications to the invention to adapt it to various usages and conditions. As such, these changes and modifications are properly, equitably, and intended to be, within the full range of equivalence of the following claims.

What is claimed is:

1. A method for terminally sterilizing a transdermal device adapted to deliver an influenza vaccine, comprising the steps of:
providing a microprojection member having a plurality of microprojections that are adapted to pierce the stratum comeum of a patient having a biocompatible coating disposed on said microprojection member, said coating being formed from a coating formulation having at least one influenza vaccine disposed thereon; and
exposing said microprojection member to radiation selected from the group consisting of gamma radiation and e-beam, wherein said radiation is sufficient to reach a desired sterility assurance level.

2. The method of claim 1, further comprising the step of sealing said microprojection member inside packaging adapted to control environmental conditions surrounding said microprojection member.

3. The method of claim 2, wherein said packaging comprises a foil pouch.

4. The method of claim 2, further comprising the step of sealing a desiccant inside said packaging.

5. The method of claim 2, further comprising the step of mounting said microprojection member on a pre-dried retainer ring prior to sealing said microprojection member inside said packaging.

6. The method of claim 4, further comprising the step of mounting said microprojection member on a pre-dried retainer ring prior to sealing said microprojection member inside said packaging.

7. The method of claim 2, further comprising the step of purging said packaging with an inert gas prior to sealing said microprojection member.

8. The method of claim 7, wherein said inert gas comprises nitrogen.

9. The method of claim 2, wherein said step of exposing said microprojection member to radiation occurs at approximately −78.5-25° C.

10. The method of claim 2, wherein said step of exposing said microprojection member to radiation occurs at an ambient temperature.

11. The method of claim 2, wherein said step of exposing said microprojection member to radiation comprises delivering in the range of approximately 5 to 50 kGy.

12. The method of claim 2, wherein said step of exposing said microprojection member to radiation comprises delivering approximately 7 kGy.

13. The method of claim 2, wherein said step of exposing said microprojection member to radiation comprises delivering approximately 21 kGy.

14. The method of claim 2, wherein said step of exposing said microprojection member to radiation comprises delivering radiation at a rate of greater than approximately 3.0 kGy/hr.

15. The method of claim 2, wherein said sterility assurance level is 10-6.

16. The method of claim 2, further comprising the step of adding an antioxidant to said coating formulation.

17. A method for terminally sterilizing a transdermal device adapted to deliver an influenza vaccine, comprising the steps of:
providing a microprojection member having a plurality of microprojections that are adapted to pierce the stratum corneum of a patient having a biocompatible coating disposed on said microprojection member, said coating being formed from a coating formulation having at least one influenza vaccine disposed thereon;
sealing said microprojection member with a desiccant inside packaging purged with nitrogen and adapted to control environmental conditions surrounding said microprojection member; and
exposing said microprojection member to radiation selected from the group consisting of gamma radiation and e-beam radiation, wherein said radiation is sufficient to reach a desired sterility assurance level.

18. The method of claim 17, further comprising the step of mounting said microprojection member on a pre-dried retainer ring prior to sealing said microprojection member inside said packaging.

19. The method of claim 17, wherein said step of exposing said microprojection member to radiation comprises delivering a dose of radiation in the range of approximately 7-21 kGy.

20. The method of claim 19, wherein said step of exposing said microprojection member to radiation occurs at an ambient temperature.

21. The method of claim 17, wherein said influenza vaccine retains at least approximately 96% of initial purity.

22. The method of claim 21, wherein said influenza vaccine retains at least approximately 98% of initial purity.

23. A method for terminally sterilizing a transdermal device adapted to deliver an influenza vaccine, comprising the steps of:
providing a microprojection member having a plurality of microprojections that are adapted to pierce the stratum comeum of a patient having a biocompatible coating disposed on said microprojection member, said coating being formed from a coating formulation having at least one influenza vaccine disposed thereon;
sealing said microprojection member inside packaging purged with an inert gas and adapted to control environmental conditions surrounding said microprojection member; and
exposing said microprojection member to e-beam radiation, wherein said radiation is sufficient to reach a desired sterility assurance level.

24. A method for terminally sterilizing a transdermal device adapted to deliver an influenza vaccine, comprising the steps of:
providing a microprojection member having a plurality of microprojections that are adapted to pierce the stratum corneum of a patient having a biocompatible coating disposed on said microprojection member, said coating being formed from a coating formulation having at least one influenza vaccine disposed thereon;
placing said microprojection member inside packaging adapted to control environmental conditions;
reducing moisture content inside said packaging;
sealing said microprojection member with said packaging; and
exposing said microprojection member to radiation selected from the group consisting of gamma radiation and e-beam, wherein said radiation is sufficient to reach a desired sterility assurance level.

25. A transdermal system, adapted to deliver an influenza vaccine, comprising:
a microprojection member including a plurality of microprojections that are adapted to pierce the stratum corneum of a patient having a biocompatible coating disposed on said microprojection member, said coating being formed from a coating formulation having at least one influenza vaccine disposed thereon; and
packaging purged with an inert gas and adapted to control environmental conditions sealed around said microprojection member;
wherein said sealed package has been exposed to radiation to sterilize the microprojection member.

26. The system of claim 25, further comprising a desiccant sealed inside said packaging with said microprojection member.

27. The system of claim 25, wherein said microprojection member is mounted on a pre-dried retainer ring.

28. The system of claim 25, wherein said packaging is purged with nitrogen.

29. The system of claim 25, wherein said packaging comprises a foil pouch.

30. The system of claim 25, wherein said influenza vaccine comprises a trivalent influenza vaccine.

31. A transdermal system, adapted to deliver an influenza vaccine, comprising:
a microprojection member including a plurality of microprojections that are adapted to pierce the stratum comeum of a patient;
a hydrogel formulation having at least one influenza vaccine, wherein said hydrogel formulation is in communication with said microprojection member; and
packaging purged with an inert gas and adapted to control environmental conditions sealed around said microprojection member;
wherein said sealed package has been exposed to radiation to sterilize the microprojection member.

32. A transdermal system, adapted to deliver an influenza vaccine, comprising:
a microprojection member including a plurality of microprojections that are adapted to pierce the stratum corneum of a patient;
a solid film disposed proximate said microprojection member, wherein said solid film is made by casting a liquid formulation comprising at least one influenza vaccine, a polymeric material, a plasticizing agent, a surfactant and a volatile solvent; and
packaging purged with an inert gas and adapted to control environmental conditions sealed around said microprojection member;
wherein said sealed package has been exposed to radiation to sterilize the microprojection member.
